(19) United States
(12) Patent Application Publication
(10) Pub. No.: US 2002/0048581 A1
(43) Pub. Date: Apr. 25, 2002

(54) Modulation of nitric oxide synthase by PKC

(76) Inventor: George L. King, Dover, MA (US)

Correspondence Address: LOUIS MYERS, Fish & Richardson P.C., 225 Franklin Street, Boston, MA, 02110-2804, US

(21) Appl. No.: 09/907,012

(22) Filed: Jul. 17, 2001

Related U.S. Application Data
(60) Provisional application No. 60/219,246, filed on Jul. 18, 2000.

(51) Int. Cl. A61K 48/00; A61K 39/395; A61K 38/17

(52) U.S. Cl. 424/146.1; 514/12; 514/44

(57) ABSTRACT

Featured are methods of modulating endothelial NOS (eNOS) expression, e.g., insulin-stimulated eNOS expression, by modulating PKCβ. The methods are useful in the treatment of insulin-related disorders, e.g., hypertension.

### RELATED APPLICATIONS

This application claims the benefit of U.S. provisional application No. 60/219,246, filed on Jul. 18, 2000, the contents of which is incorporated herein by reference in its entirety.

### BACKGROUND

Insulin has multiple physiological effects on vascular tissues, such as vasodilation, which may be endothelial cell dependent and can be inhibited by inhibitors of nitric oxide synthase (NOS) (Feener et al. Lancet. 1997;350(suppl 1):SI9-SI13; Scherrer et al. Circulation. 1997;96:4104-4113; Baron et al. Am J Physiol. 1996;271:E1067-E1072; Yki-Jarvinen et al. Diabetologia. 1998;41:369-379; Steinberg et al. J Clin Invest. 1994;94:1172-1179; Utriainen et al. Diabetologia. 1996;39:1477-1482). Insulin has been suggested to increase the production of NO acutely in cultured endothelial cells within a few minutes, indicating an activation of NOS via the insulin receptors (Zeng et al. J Clin Invest. 1996;98:894-898).

### SUMMARY OF THE INVENTION

The inventors have discovered that insulin can regulate (e.g., chronically) the expression of eNOS, e.g., by increasing eNOS mRNA levels, e.g., in endothelial cells and microvessels. Further, the inventors have found that activation of PKC, e.g., PKCβ, e.g., PKCβ1, inhibits insulin-stimulated eNOS expression. The activation of PKC in vascular tissues, e.g., as seen in insulin related disorders, e.g., diabetes or insulin resistance and its associated conditions, e.g., hypertension, atheroscleorsis, ischemia, coronary heart disease, glucose intolerance, obesity, dyslipidemia (increased triglycerides, decreased HDL, increased small dense LDL), may inhibit eNOS expression thereby leading to endothelial dysfunctions in these pathological states. Accordingly, one aspect of the invention features a method of treating an insulin related disorder, e.g., diabetes, insulin resistance, hypertension, glucose intolerance, atherosclerosis, ischemia, vascular disease, or dyslipidemia, by modulating PKC, e.g., PKC β, e.g., PKCβ1, or by modulating PI3 kinase activity, thereby modulating eNOS expression (e.g., eNOS mRNA levels, mRNA stability, mRNA transcription rate) to treat the disorder.

In one aspect, the invention features a method of modulating eNOS in a cell, tissue, or subject (e.g., a subject having an insulin related disorder described herein, or a cell or tissue from a subject having an insulin related disorder described herein). The method includes modulating PKC, e.g., PKCβ (e.g., PKCβ1). Modulating PKCβ can modulate eNOS mRNA levels, e.g., eNOS mRNA half-life and/or eNOS mRNA transcription rate. Preferably, eNOS expression is modulated for at least 1 hour, e.g., for 2 hours, 4 hours, 6 hours, 12 hours, 24 hours, 48 hours, or longer. The subject in any method described herein can be a human or a non-human animal, e.g., an experimental animal, e.g., a rodent, e.g., a rodent model for an insulin related disorder, e.g., an obese rodent, e.g., a Zucker rat, a fructose fed rodent, the Israeli sand rat (Psammomys obesus).

In a preferred embodiment, the PKCβ is a PKCβ1.

In a preferred embodiment, PKC activity is inhibited, e.g., through the use of a PKC inhibitory agent, preferably a PKC β (e.g., a PKC β1) inhibitory agent. The agent can be one or more of: a small molecule which inhibits PKC activity; a PKC binding protein which binds to PKC but does not activate the enzyme; an antibody that specifically binds to the PKC protein, e.g., an antibody that disrupts PKC's catalytic activity or an antibody that disrupts the ability of upstream activators to activate PKC; a PKC nucleic acid molecule which can bind to a cellular PKC nucleic acid sequence, e.g., mRNA, and inhibit expression of the protein, e.g., an antisense molecule or PKC ribozyme; an agent which decreases PKC gene expression, e.g., a small molecule which binds the promoter of PKC. In another preferred embodiment, PKC is inhibited by decreasing the level of expression of an endogenous PKC gene, e.g., by decreasing transcription of the PKC gene. In a preferred embodiment, transcription of the PKC gene can be decreased by: altering the regulatory sequences of the endogenous PKC gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, an inhibitor of PKC β is administered to the cell, tissue, or subject. The inhibitor can be an inhibitory PKCβ antibody, a PKCβ antisense nucleic acid (e.g., an antisense RNA or ribozyme), an inhibitory PKCβ binding peptide (e.g., a peptide that inhibits PKCβ activity), or an inhibitory PKCβ binding small molecule. For example, the inhibitor can be LY333531.

In a preferred embodiment, the subject exhibits an insulin related disorder, e.g., insulin resistance, diabetes, hypertension, or another insulin related disorder described herein.

In another embodiment, PKC activity, e.g., PKC β activity (e.g., PKC β1 activity) is increased, e.g., by administering an agent that increases PKC activity. The agent that increases PKC activity can be one or more of the following: a small molecule which stimulates PKC activity, e.g., PMA; a PKC polypeptide or a functional fragment or analog thereof; a nucleotide sequence encoding a PKC polypeptide or functional fragment or analog thereof; an agent which increases PKC nucleic acid expression; e.g., a small molecule which binds to the promoter region of PKC. In a preferred embodiment, PKC levels are increased by administering, e.g., introducing, a nucleotide sequence encoding a PKC polypeptide or functional fragment or analog thereof, into a particular cell, e.g., an endothelial cell, in the subject. The nucleotide sequence can be a genome sequence or a cDNA sequence. The nucleotide sequence can include: a PKC coding region; a promoter sequence, e.g., a promoter sequence from a PKC gene or from another gene; an enhancer sequence; untranslated regulatory sequences, e.g., a 5′untranslated region (UTR), e.g., a 5′UTR from a PKC gene or from another gene, a 3′UTR, e.g., a 3′UTR from a PKC gene or from another gene; a polyadenylation site; an insulator sequence. In another preferred embodiment, the level of PKC protein is increased by increasing the level of expression of an endogenous PKC gene, e.g., by increasing transcription of the PKC gene. In a preferred embodiment, transcription of the PKC gene is increased by: altering the regulatory sequence of the endogenous PKC gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the PKC gene to be transcribed more efficiently. Preferably, the agent increases PKCβ activity.

In one aspect, the invention features a method of increasing eNOS, e.g., eNOS expression, e.g., eNOS mRNA levels, in a cell, tissue, or subject. The method includes inhibiting PKCβ, e.g., PKCβ1.

In a preferred embodiment, a PKCβ inhibitor described herein is administered to the cell, tissue, or subject. E.g., the inhibitor can be an inhibitory PKCβ antibody, a PKCβ antisense nucleic acid (e.g., an antisense RNA or ribozyme), an inhibitory PKCβ binding peptide (e.g., a peptide that inhibits PKCβ activity), or an inhibitory PKCβ binding small molecule. For example, the inhibitor can be LY333531.

In a preferred embodiment, eNOS mRNA levels are increased. For example, mRNA transcription rate or half-life is increased.

In a preferred embodiment, the subject has an insulin related disorder, or the cell or tissue are derived from a subject that has an insulin related disorder, e.g., an insulin related disorder described herein.

In a preferred embodiment, the insulin related disorder is hypertension.

In a preferred embodiment, the insulin related disorder is diabetes.

In a preferred embodiment, the insulin related disorder is insulin resistance.

In another aspect, the invention features a method of increasing eNOS in a cell, tissue, or subject, e.g., a subject exhibiting an insulin related disorder, or a cell or tissue therefrom). The method includes increasing PI3 kinase activity.

An agent which increases PI3-kinase activity can be one or more of the following: a small molecule which activates PI3kinase; a PI3kinase polypeptide or a functional fragment or analog thereof; a nucleotide sequence encoding a PI3kinase polypeptide or functional fragment or analog thereof; an agent which increase PI3-kinase nucleic acid expression, e.g., a small molecule which binds to the promoter region of PI3 kinase. In a preferred embodiment, PI3-kinase levels are increased by administering, e.g., introducing, a nucleotide sequence encoding a PI3-kinase polypeptide or functional fragment or analog thereof, into a particular cell, e.g., an endothelial cell, in the subject. The nucleotide sequence can be a genome sequence or a cDNA sequence. The nucleotide sequence can include: a PI3-kinase coding region; a promoter sequence, e.g., a promoter sequence from a PI3 kinase gene or from another gene; an enhancer sequence; untranslated regulatory sequences, e.g., a 5′untranslated region (UTR), e.g., a 5′UTR from a PI3kinase gene or from another gene, a 3′UTR, e.g., a 3′UTR from a PI3-kinase gene or from another gene; a polyadenylation site; an insulator sequence. In another preferred embodiment, the level of PI3kinase protein is increased by increasing the level of expression of an endogenous PI3-kinase gene, e.g., by increasing transcription of the PI3-kinase gene. In a preferred embodiment, transcription of the PI3-kinase gene is increased by: altering the regulatory sequence of the endogenous PI3 kinase gene, e.g., by the addition of a positive regulatory element (such as an enhancer or a DNA-binding site for a transcriptional activator); the deletion of a negative regulatory element (such as a DNA-binding site for a transcriptional repressor)and/or replacement of the endogenous regulatory sequence, or elements therein, with that of another gene, thereby allowing the coding region of the PI3-kinase gene to be transcribed more efficiently.

In a preferred embodiment, eNOS mRNA levels are increased.

In a preferred embodiment, the subject has an insulin related disorder.

In preferred embodiments, the subject can have at least one of: diabetes, insulin resistance, or hypertension. In a preferred embodiment, PI3 kinase activity is increased to treat hypertension.

In yet another aspect, the invention features a method of treating hypertension in a subject. The method includes identifying a subject in need of treatment for hypertension; and administering a PKCβ inhibitor, e.g., LY333531. The PKCβ inhibitor, e.g., LY333531, increases eNOS expression in a tissue of the subject, thereby treating hypertension. In a preferred embodiment, eNOS expression is increased at least 10% compared to a control (e.g., a subject who has not been administered a PKCβ inhibitor, e.g., a subject who has not been administered LY333531). Preferably, eNOS expression is increased at least 20, 30, 40, 50, 60, 70, 80, 90, 100, 150, or 200% or more, compared to a control. The method can include the step of evaluating the subject for hypertension before and/or after the administration of the PKCβ inhibitor (e.g., LY333531).

In another aspect, the invention features a kit for treating hypertension in a subject. The kit includes a pharmaceutical composition that includes a PKC β inhibitor. The kit can also include instructions for using the pharmaceutical composition to treat hypertension. For example, the instructions can include instructions regarding, e.g., the mode, time, and/or dosage of administration of the PKCβ inhibitor to the subject.

In a preferred embodiment, the PKC β inhibitor is LY333531.

In a preferred embodiment, the subject is a human.

In another aspect, the invention features a method of screening for agents that can inhibit an effect or symptom of an insulin related disorder, e.g., an insulin related disorder described herein. The method includes (1) providing a cell (e.g., an endothelial cell), a tissue (e.g., a vascular tissue, e.g., a microvascular tissue), or a subject (e.g., an experimental animal, e.g., an animal model for an insulin related disorder); (2) contacting the cell, tissue, or subject with a test agent; and (3) evaluating the effect of the test agent on any of: PKC activity, e.g., PKCβ activity, eNOS activity; eNOS expression, e.g., eNOS mRNA levels. The method can include evaluating the effect of the test agent on the cell, tissue, or subject, compared to a control, e.g., a cell, tissue, or subject that has not been exposed to the test agent.

In one embodiment, the method includes administering insulin to the cell, tissue, or subject in the presence or absence of a test agent, and evaluating the effect on any of: PKC activity, e.g., PKCβ activity, eNOS activity; eNOS expression, e.g., eNOS mRNA levels.

In one embodiment, the method can further include administering the test agent to an animal, e.g., an animal model for an insulin related disorder, e.g., an animal model for hypertension or another disorder described herein.

In another aspect, the invention features a method of determining if a subject, e.g., a human, is at risk for hypertension. The method includes: evaluating a PKCβ activity in the subject, e.g., in a cell or tissue of the subject, and comparing the PKCβ activity in the cell or tissue of the subject to a control, e.g., a cell or tissue from a non-hypertensive subject. A higher PKCβ activity in the subject compared to a control indicates that the subject has or is at risk for hypertension. The method can also include evaluating the subject for hypertension or a symptom of hypertension. A methods of evaluating PKC activity is described in the Examples below. Other PKC assay methods are known in the art.

The terms “peptides”, “proteins”, and “polypeptides” are used interchangeably herein.

The term “small molecule”, as used herein, includes peptides, peptidomimetics, or non-peptidic compounds, such as organic molecules, having a molecular weight less than 2000, preferably less than 1000.

As used herein, “preventing or treating”, e.g., hypertension, means the application or administration of a therapeutic agent, e.g., a PKC β inhibitor, e.g., LY333531, to a subject who has or is at risk for a disorder, e.g., an insulin related disorder, e.g., hypertension, with the purpose to reduce, improve, alleviate, alter, remedy, ameliorate, or affect, the disorder or a symptom of the disorder. A treatment, e.g., a pharmaceutical composition described herein, can be administered to the subject by the subject himself or herself, or by another person, e.g., a health care provider.

Other embodiments are within the following description and the claims.

### DETAILED DESCRIPTION

The inventors have found that the β isoform of PKC (PKCβ) can selectively modulate the effect of insulin on eNOS expression, e.g., on eNOS mRNA levels. This finding was surprising in that the PKCβ isoform is expressed to a lesser extent than other PKC isoforms in endothelial cells (Kent et al. Circ Res. 1995;77:231-238).

As shown in the Examples presented herein, the inhibitory effect of the PKCβ isoform on eNOS mRNA level was directly confirmed through the overexpression of the PKCβ isoform in endothelial cells with the use of adenoviral vectors containing full-length DNA of the PKCβ₁ isoform. The inhibitory effect of PKC activation on eNOS expression is specific to insulin because the stimulating effect of lysophosphatidylcholine (LPC) on eNOS was not affected. Rapid PKC activation induced by phorbol esters caused inhibition of insulin-stimulated PI-3 kinase activity and eNOS mRNA expression. eNOS expression was increased by the long-term incubation of PMA and by PKC inhibitors, both of which reduce PKC activities in endothelial cells.

These findings confirm that PKC inhibition increases eNOS mRNA in bovine aortic endothelial cells (BAECs). The findings that both general PKC inhibitor GFX and specific PKCβ isoform inhibitor LY333531 increased basal eNOS levels indicate that PKC activities can regulate eNOS mRNA levels in endothelial cells. The use of the PKCβ isoform inhibitor LY333531 (20 nmol/L, a concentration that selectively inhibited the PKCβ isoform) indicated that the activation of PKCβ isoform has a selective effect on eNOS expression. Thus, eNOS expression can be modulated by modulation of PKCβ, e.g., in the treatment of an insulin related disorder, e.g., an insulin related disorder described herein.

Protein Kinase C

Protein kinase C (PKC) is a membrane-associated enzyme that is regulated by a number of factors, including membrane phospholipids, calcium, and membrane lipids such as diacylglycerols that are liberated in response to the activities of phospholipases (Bell et al. J. Biol. Chem. 1991. 266:4661-4664; Nishizuka, Science 1992. 258:607-614. The protein kinase C isozymes, alpha, beta(β)-1, beta-2 and gamma, require membrane phospholipid, calcium and diacylglycerol/phorbol esters for full activation. The delta, epsilon, eta, and theta forms of PKC are calcium-independent in their mode of activation. The zeta and lambda forms of PKC are independent of both calcium and diacylglycerol and are believed to require only membrane phospholipid for their activation. PKC- and isozyme-specific (e.g., PKC β specific) modulators are described, e.g., in Goekjian et al. Current Medicinal Chemistry, 1999, 6:877-903; Way et al., Trends Pharmacol Sci, 2000, 21:181-7, and in U.S. Pat. No. 5,843,935.

The invention also provides methods for identifying modulators, i.e., candidate or test compounds or agents (e.g., proteins, peptides, peptidomimetics, peptoids, small molecules or other drugs) which have stimulatory or inhibitory effect on, for example, the expression or activity of PKC β, thereby modulating eNOS expression, e.g., eNOS mRNA levels. Compounds thus identified can be used to modulate the activity of PKC β, e.g., PKCβ1, in a method described herein.

Generation of Analogs: Production of Altered DNA and Peptide Sequences by Random Methods

Amino acid sequence variants of a protein, e.g., a PKC βagonist or antagonist, can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants, e.g., screening for PKC β modulating activity, are elsewhere herein.)

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Analogs: Production of Altered DNA and Peptide Sequences by Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. (1978) USA, 75: 5765).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-through-put Methods for Screening Libraries of Peptide Fragments or Homologs

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with a PKC, e.g., PKC β, e.g., PKC β1. These may include agonists, superagonists, and antagonists of PKC, PKC β, or PKC β1. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein. e.g., a PKC, e.g., a PKC β., e.g., PKC β1 molecule or a fragment thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869).

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382). A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Secondary Screens

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest (e.g., PKC, PKC β, or PKC β 1) and a ligand (e.g., a PKC substrate) can be used to identify antagonists from a group of peptide fragments isolated though one of the primary screens described above.

Therefore, methods for generating fragments and analogs and testing them for activity are known in the art. Once the core sequence of interest is identified, it is routine to perform for one skilled in the art to obtain analogs and fragments.

Peptide Mimetics

The invention also provides for reduction of the protein binding domains of the subject polypeptides, e.g., PKC, e.g., PKC β (e.g., PKC β1), to generate mimetics, e.g. peptide or non-peptide agents. See, for example, “Peptide inhibitors of human papillomavirus protein binding to retinoblastoma gene protein” European patent applications EP 0 412 762 and EP 0 031 080.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Antibodies

The invention also includes antibodies specifically reactive with a PKC described herein. Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

PKC (e.g., PKC β, preferably PKC β1), or a portion or fragment thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant PKC peptide, or a chemically synthesized PKC peptide or anagonist. See, e.g., U.S. Pat. No. 5,460,959; and co-pending U.S. application Ser. Nos. 08/334,797; 08/231,439; 08/334,455; and 08/928,881 which are hereby expressly incorporated by reference in their entirety. The nucleotide and amino acid sequences of PKC isozymes described herein are known. The preparation can further include an adjuvant, such as Freund's complete or incomplete adjuvant, or similar immunostimulatory agent. Immunization of a suitable subject with an immunogenic PKC preparation induces a polyclonal anti-PKC antibody response.

Antibodies to PKC (preferably PKC β, e.g., PKC β1) or fragments thereof, can be used to inhibit the levels of such a component, thereby increasing NOS activity. Examples of antibody fragments include F(v), Fab, Fab′ and F(ab′)₂ fragments which can be generated by treating the antibody with an enzyme such as pepsin. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. 5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against a PKC described herein can be made using standard techniques. For example, human monoclonal antibodies can be generated in transgenic mice or in immune deficient mice engrafted with antibody-producing human cells. Methods of generating such mice are describe, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with aPKC described herein or an antigenic peptide thereof and splenocytes from these immunized mice can then be used to create hybridomas. Methods of hybridoma production are well known.

Human monoclonal antibodies against a PKC described herein can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind a PKC, can be mutated, by for example using randomly altered mutagenized oligonucleotides, to generate a library of mutated variable regions which can then be screened to bind to a PKC. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds a PKC described herein. In a preferred embodiment, the primary screening of the library involves panning with an immobilized PKC described herein and display packages expressing antibodies that bind immobilized PKC described herein are selected.

Antisense Nucleic Acid Sequences

Nucleic acid molecules which are antisense to a nucleotide encoding a PKC described herein, e.g., PKC β, e.g., PKC β1, can be used as an agent which inhibits expression of the PKC. An “antisense” nucleic acid includes a nucleotide sequence which is complementary to a “sense” nucleic acid encoding the component, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the component can be used.

The coding strand sequences encoding PKC isozymes described herein are known. Given the coding strand sequences encoding these isozymes, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Administration

An agent which modulates the level of expression of a PKC described herein can be administered to a subject by standard methods. For example, the agent can be administered by any of a number of different routes including intravenous, intradermal, subcutaneous, oral (e.g., inhalation), transdermal (topical), and transmucosal. In one embodiment, the PKC modulating agent can be administered orally. In another embodiment, the agent is administered by injection, e.g., intramuscularly, or intravenously.

The agent which modulates protein levels, e.g., nucleic acid molecules, polypeptides, fragments or analogs, modulators, and antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a PKC β polypeptide or anti-PKC β antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Gene Therapy

The nucleic acids described herein, e.g., a nucleic acid encoding a PKC isozyme described herein, or an antisense nucleic acid, can be incorporated into gene constructs to be used as a part of a gene therapy protocol to deliver nucleic acids encoding either an agonistic or antagonistic form of a PKC described herein, e.g., a PKC β. The invention features expression vectors for in vivo transfection and expression of PKC described herein in particular cell types so as to reconstitute the function of, or alternatively, antagonize the function of the component in a cell in which that polypeptide is misexpressed. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g. a cDNA, encoding a PKC described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a PKC described herein in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding a PK described herein (e.g., a PKC β) can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

A PKC described herein can also be increased in a subject by introducing into a cell, e.g., an endothelial cell, a nucleotide sequence that modulates the production of PKC, e.g., a nucleotide sequence encoding a PKC polypeptide or functional fragment or analog thereof, a promoter sequence, e.g., a promoter sequence from a PKC gene or from another gene; an enhancer sequence, e.g., 5′ untranslated region (UTR), e.g., a 5′ UTR from a PKC gene or from another gene, a 3′ UTR, e.g., a 3′ UTR from a PKC gene or from another gene; a polyadenylation site; an insulator sequence; or another sequence that modulates the expression of PKC, e.g., PKC β, e.g., PKC β 1. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient).

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of secondary cells which have been passaged one or more times.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding a PKC described herein, e.g., PKC β, e.g., PKC β 1, or an agonist or antagonist thereof, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No.: 5,641,670, the contents of which are incorporated herein by reference. The transfected primary or secondary cells may also include DNA encoding a selectable marker which confers a selectable phenotype upon them, facilitating their identification and isolation.

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration, all of which are routine in the art.

Transfected primary or secondary cells undergo sufficient number doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from an insulin related disorder is a candidate for implantation of cells producing an antagonist of PKC β described herein.

An immunosuppressive agent e.g., drug, or antibody, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541′ Widner et al. (1992) n. Engl. J. Med. 327:1556). Dosage values may vary according to factors such as the disease state, age, sex, and weight of the individual.

### EXAMPLES

### Example 1

Effect of Insulin on mRNA and Protein Levels of eNOS

One of the important vascular actions of insulin is its vasodilatory effect, which is associated with nitric oxide (NO) production, either from endothelial cells or from perivascular neuronal cells. Insulin and IGF-1 increased NO production in endothelial cells in <1 minute (Zeng et al. J Clin Invest. 1996;98:894-898; Tsukahara et al. Kidney Int. 1994;45:598-604), suggesting that insulin can directly activate eNOS because protein and mRNA levels of eNOS could not increase so rapidly. However, the acute effect of insulin on NO production cannot account for all of the vasodilatory effects of insulin in vivo, because some physiological studies have reported that the vasodilatory effect of insulin continues to increase even after 7 hours of infusion (Utriainen et al. Diabetologia 1996;39:1477-1482), suggesting that the vasodilatory effect of insulin has a sustained component that requires several hours of stimulation when near-physiological concentrations of insulin are used.

Insulin (100 nmol/L) significantly augmented the eNOS mRNA expression of eNOS at 1 hour by 33±9%, reached 71±21% at 6 hours, and attained a maximum of 2-fold at 12 hours, which was maintained for 24 hours. Expression of eNOS mRNA responded to insulin with a significant increase even at 0.1 nmol/L. At 10 nmol/L insulin, eNOS mRNA level was significantly increased by 50±12%, and a maximum effect of 2-fold was attained at 100 nmol/L. Therefore, stimulation with 100 nmol/L insulin with an incubation time of 6 hours IGF-1 (25 nmol/L) also increased the eNOS mRNA level by 47±10% in human umbilical endothelial cells. The addition of alpha-IR3, an IGF-1 receptor specific antibody (1 μg/mL), inhibited the effect of IGF-1 by 60% but did not decrease the effect of insulin. Insulin also increased eNOS protein levels at 6 hours by 43±16% and reached a maximum of 2-fold at 24 hours, which was maintained for 36 hours. Thus, insulin can modulate eNOS expression chronically both in vitro and in vivo.

The effect of insulin on eNOS mRNA levels was observed between 0.1 and 100 nmol/L, which corresponds closely to the range of binding and activation of insulin receptors in the endothelial cells and to the physiological levels of insulin in the plasma, while the rapid effect of insulin on NO production in cultured human umbilical endothelial cells required pharmacological insulin concentrations of 10 to 10 000 nmol/L (Zeng et al. J Clin Invest. 1996;98:894-898). Thus, the signaling pathways of insulin action on eNOS mRNA appear to involve mainly the insulin receptors, because the maximal effect on eNOS mRNA level was attained with <100 nmol/L, a concentration of insulin shown to bind minimally to IGF-1 receptors in endothelial cells.¹ In addition, the effect of insulin on eNOS mRNA was not prevented by inhibitory antibodies to IGF-1 receptors.

### Example 2

Effect of PI-3 Kinase Inhibitors Wortmannin and LY294002 on Expression of eNOS

The acute effect of insulin on NO production in endothelial cells was reported to be inhibited by wortmannin, a PI-3 kinase inhibitor. To determine whether PI-3 kinase activation could be increasing mRNA expression and protein levels of eNOS, 2 structurally different PI-3 kinase inhibitors, wortmannin (100 nmol/L) and LY294002 (50 nmol/L), were preincubated with BAECs before the addition of insulin (100 nmol/L). Insulin increased the mRNA level of eNOS by 58±20% compared with control, but the effect of insulin was inhibited by preincubation with wortmannin. Similar to eNOS mRNA levels, insulin significantly increased the eNOS protein level by 74±9%, which was completely inhibited by the addition of wortmannin.

The pretreatment of BAECs with another PI-3 kinase inhibitor, LY294002 (50 nmol/L), completely inhibited the induction of eNOS mRNA expression by insulin. Unlike wortmannin, LY294002 significantly decreased the basal mRNA expression of eNOS without insulin treatment by 30±4%. Correspondingly, LY294002 inhibited the increases in eNOS protein levels stimulated by insulin and decreased the basal eNOS protein level by 72±5%.

Insulin (100 nmol/L) significantly increased NOS activity from 115±9 to 176±7 pmol/mg protein/min after 24 hours (P=0.01, n=6). Preincubation with wortmannin (100 nmol/L) for 15 minutes significantly decreased insulin-induced NOS activity to 123±13 pmol/mg protein/min, but the basal levels of NOS activity were unchanged.

### Example 3

Effect of PMA on Insulin-induced eNOS mRNA Expression and PI-3 Kinase Activities

Because PKC activation is observed in the vascular tissue in diabetes and may regulate eNOS in BAECs (Ohara et al. Hypertension. 1995;25:415-420), the actions of PMA, a PKC agonist, on eNOS expression were studied. In time course experiments, PMA (100 nmol/L) did not change the eNOS mRNA level for the initial 6 hours but significantly increased the expression of eNOS mRNA after 12 and 24 hours of incubation by 66±11% and 105±14%, respectively. In contrast, when BAECs were preincubated with PMA for 30 minutes, the effect of insulin on eNOS mRNA levels was inhibited (14±13%).

Because insulin may increase NO production via activation of PI-3 kinase through the tyrosine phosphorylation of its receptors and IRS, the effects of PKC activation on the insulin induction of eNOS expression and PI-3 kinase activity were examined in parallel. Insulin significantly increased IRS-2-associated PI-3 kinase activity by 5.4±0.4-fold. When BAECs were preincubated with PMA (100 nmol/L) for 30 minutes, insulin-induced IRS-2-associated PI-3 kinase activity was mostly inhibited. However, the basal PI-3 kinase activity was not changed with PMA treatment.

### Example 4

Effect of PKC Inhibitors on eNOS mRNA Expression

The exposure of BAECs to the PKC inhibitor GFX (5 μmol/L) without insulin for 6 hours increased the expression of eNOS mRNA by 38±10%. The expression of eNOS mRNA was greater in cells exposed to both insulin and GFX (by 76±20% compared with control cells or those incubated with either insulin or GFX alone). As hyperglycemia may preferentially activate PKCβ isoforms in the vascular cells (Ishii et al. Science. 1996;272:728-731), the possibility that the PKCβ isoform could also have a role in regulation of the activation by insulin of PI-3 kinase and eNOS expression, the effect of LY333531 (20 nmol/L), a PKCβ isoform inhibitor, was characterized. The addition of LY333531 also increased eNOS mRNA expression by 60±14%, which is similar to insulin or GFX alone. LY333531 and insulin together did not have a significant additive effect, suggesting that the PKC effect is due mainly to PKC β.

### Example 5

Effect of Overexpression of PKCβ Isoform on Insulin-induced eNOS mRNA Level

To determine directly whether the PKCβ isoform can regulate the effect of insulin on eNOS expression, the PKCβ₁ isoform was overexpressed in BAECs through the use of replication-deficient adenovirus containing cDNA of the PKCβ₁ isoform. Compared with control cells infected with adenovirus containing the β-Gal gene, cells infected with adenovirus containing the PKCβ₁ gene had a 50-fold increase in the protein for the PKCβ₁ isoform. Total PKC activities were also increased by 11- and 7-fold in the cytosol and membrane fractions, respectively.

Insulin (100 nmol/L) enhanced eNOS mRNA expression in BAECs with or without infection with adenovirus containing only β-Gal by as much as 2-fold. In contrast, insulin did not increase eNOS mRNA levels in cells infected with adenovirus containing the PKCβ₁ isoform. The expression of eNOS was not changed by overexpression of the PKCβ₁ isoform at the basal level. In contrast, LPC (100 μmol/L), which is known to stimulate eNOS, increased eNOS mRNA levels by 5- and 4.5-fold in control and adenovirus-containing β-Gal cells, respectively. In BAECs infected with the adenoviral-PKCβ₁ isoform, LPC increased eNOS mRNA by 4-fold, which was not significantly different from controls.

### Example 6

Effect of Insulin on eNOS mRNA Level in Vascular Stroma Isolated From Epididymal Fat Pads of Zucker Fatty and Lean Rats

To determine whether insulin can also change eNOS expression in vascular tissue, we characterized eNOS mRNA levels in vascular stroma isolated from Zucker lean and fatty insulin-resistant rats, a model of insulin resistance (Shimabukuro et al. J Biol Chem. 1998;273:3547-3550). The expression of eNOS mRNA with or without insulin (100 nmol/L) for 6 hours in the vascular stroma isolated from insulin-resistant models (Zucker fatty rats) showed that the basal levels of eNOS mRNA expression were significantly decreased to 29±5% of vascular stroma derived from Zucker lean rats. The contents of vascular stroma in both preparations were found to be similar through the use of immunostaining with factor VIII antibodies and immunoblotting with antibodies to smooth muscle cell alpha-actin. Moreover, insulin increased eNOS mRNA levels by 50±16% in the vascular stroma from the Zucker lean rats but was ineffective in vascular stroma isolated from the insulin-resistant rats.

The results obtained for the microvessels isolated from the Zucker fatty and lean rats support the likelihood that our findings in cultured endothelial cells have physiological meaning and that this action of insulin is blunted in insulin-resistant states. These in vivo findings are consistent with previous reports that showed the total NOS activities were decreased in the skeletal muscle and neuronal tissues of Zucker fatty rats. The basal expression of eNOS was also much lower in insulin-resistant Zucker fatty rats than in lean animals, suggesting that insulin may also modulate eNOS levels in the vessels at the basal state.

### Example 7

Methods and Materials

Cell Culture

Bovine aortic endothelial cells (BAECs) from passages 4 to 10 were isolated. Confluent cells were placed in DMEM containing 1% platelet-deprived horse serum (PDHS) for 24 hours before being studied and pretreated with the following inhibitors: phosphatidylinositol-3 (PI-3) kinase-selective inhibitors wortmannin (Sigma Chemical Co) and LY294002 (BIOMOL Research Laboratories), protein kinase C (PKC) activator phorbol-12-myristate-13-acetate (PMA) (Sigma Chemical Co), general PKC inhibitor GF109203X (GFX) (Calbiochem-Novabiochem Corp), and PKCβ isoform-selective inhibitor LY333531 (Lilly Inc). Cells were then stimulated with insulin (Sigma Chemical Co), recombinant insulin-like growth factor-1 (IGF-1) (Upstate Biotechnology), and LPC (Avanti Polar Lipid) or alpha-IR3 antibodies.

Construction of Replication-deficient Recombinant Adenovirus Containing PKCβ1 cDNA

The construction of a replication-deficient recombinant adenovirus for PKCβ1 expression was performed as described in Becker et al. Methods Cell Biol. 1994;43:161-189. Adenovirus-mediated gene transfer to confluent BAECs was performed through a 1-hour adenoviral infection of 10⁹ pfu/mL at 37° C. in DMEM containing 10% PDHS. The infected BAECs were then incubated in DMEM containing 1% PDHS for 24 hours, incubated with or without insulin (100 nmol/L) for an additional 6 hours, and harvested. AdV-CMV-PKCβ₁- or β-galactosidase (β-Gal)-infected BAECs were assessed for PKC activity and protein expression as previously described.

Isolation of Vascular Stroma from Epididymal Fat Pads of Zucker Rats

Vascular stromas were obtained from the epididymal fat pads of 12-week-old Zucker lean and fatty rats (Harlan Sprague Dawley, Inc). Epididymal fat pads were isolated, minced, and incubated with 0.2% collagenase I for 30 minutes at 37° C. Then, they were fractionated with the use of a Dounce homogenizer and centrifuged at 3000 g for 20 minutes to isolate vessels from adipocytes. Vascular stroma were washed with DMEM containing 0.2% BSA and incubated with DMEM containing 0.2% BSA with or without insulin for 6 hours at 37° C. The purity of the isolated vascular stroma was quantified through immunohistochemical staining with anti-factor VIII antibody and through immunoblotting of the stroma with antibodies to smooth muscle cell alpha-actin. Only preparations that were stained positively in more than 90% of the vessels were used.

RNA Isolation and Northern Blot Analysis

Total RNA from cultured BAECs, PKCβ₁-overexpressed BAECs, and vascular stroma from the epididymal fat pads of Zucker rats were isolated according to the guanidinium thiocyanate-phenol-chloroform method with TRI Reagent (Molecular Research Center) and solution D containing 4 mol/L guanidinium thiocyanate, 25 mmol/L sodium citrate, pH 7.0, 0.5% sarcosyl, and 0.1 mol/L 2-mercaptoethanol. Total RNA (20 μg) was fractionated and hybridized to 650-bp cDNA fragments of rat eNOS (kindly provided by Dr Mark A. Perella and Arthur M. E. Lee, Harvard School of Public Health, Boston, Mass.), which were labeled with the use of a DNA labeling system (Multiprime; Amersham Corp). The quantification of eNOS mRNA levels was performed with a Phosphorlmager (Molecular Dynamics) and normalized to 36B4 mRNA.

Immunoblot Analysis of eNOS

Cells were washed 3 times with ice-cold PBS, pH 7.4, lysed in 50 mmol/L Tris, pH 7.5, 2 mmol/L EDTA, 0.5 mmol/L EGTA, 2 mmol/LPMSF, 25 μg/mL leupeptin, 0.1 mg/mL aprotinin, 1 mmol/L dithiothreitol, 50 mmol/L NaF, and 1% Triton X-100 (Sigma Chemical Co); scraped from the dish; rotated for 1 hour at 4° C.; and centrifuged for 15 minutes at 14 000 g. Protein concentrations of the supernatant were measured according to the method of Bradford and separated with the use of 6% SDS-PAGE. The membrane was incubated for 1 hour with polyclonal anti-human eNOS antibody (Transduction Laboratories) diluted in PBS containing 0.1% Tween-20 and 1% BSA, washed 3 times for 10 minutes with PBS containing 0.1% Tween-20, and incubated with 0.1 μCi/mL ¹²⁵I-protein A (Amersham Life Science, Inc). Protein levels of eNOS were quantified with a Phosphorlmager.

Assay of PI-3 Kinase Activity

After preincubation with or without 100 nmol/L PMA for 30 minutes, BAECs were stimulated with insulin (100 nmol/L) for 5 minutes. Cells were processed as described previously for this assay. Aliquots of proteins from the supernatant were inununoprecipitated with 10 μL/ml anti-alpha-insulin receptor substrates (IRS)-2 antibodies (kindly provided by Dr Morris F. White, Joslin Diabetes Center, Boston, Mass.) for 2 hours and bound to protein A-Sepharose beads at 4° C. as described previously. The lipids were quantified with a Phosphorlmager.

Assay of NOS Activity

The amount of NOS activity produced by BAECs was measured by using an NOS Detect assay kit (Transduction Laboratories) according to the manufacturer's instructions. Briefly, BAECs were harvested in PBS containing 1 mmol/L EDTA and centrifuged at 12 000 g. The pellets were lysed in homogenization buffer containing 25 mmol/L Tris, pH 7.4, 1 mmol/L EDTA, and 1 mmol/L EGTA and centrifuged at 12 000 g. Aliquots from the supernatant were used for the measurement of NOS activity through the conversion of [³H]L-arginine to [³H]L-citrulline. Data were normalized by the amount of protein and reaction time.

PKC Activity Assay and Immunoblotting Studies

After adenoviral infection, confluent BAECs were harvested and PKC activity was measured. Briefly, PKC activities were measured according to ³²P labeling of 100 μmol/L PKC-specific peptide substrate RKRTLRRL. For inumunoblotting studies, total cell lysate (75 μg/lane) was fractionated with the use of PAGE and detected with the use of antibodies to the PKCβ₁ isoform (Santa Cruz Biotechnology, Inc). A detailed description of the method was reported previously.

Statistical Analysis

Data are expressed as mean±SEM and were analyzed with the use of the Newman-Keuls test for ANOVA for multiple comparisons. A value of P<0.05 was considered statistically significant.

All patents and references cited herein are hereby incorporated by reference in their entirety. Other embodiments are within the following claims.

What is claimed is:

1. A method of modulating endothelial cell nitric oxide synthase (eNOS) in a cell, tissue, or subject, comprising modulating a PKC β.

2. The method of claim 1, wherein the PKCβ is PKCβ1.

3. The method of claim 1, wherein modulating a PKC β comprises administering to the cell, tissue, or subject an inhibitor of PKC β.

4. The method of claim 3, wherein the inhibitor of PKC β is LY333531.

5. The method of claim 3, wherein the inhibitor of PKCβ is selected from the group of: an inhibitory PKCβ antibody, a PKCβ antisense nucleic acid, an inhibitory PKCβ binding peptide, and an inhibitory PKCβ binding small molecule.

6. The method of claim 3, wherein the subject exhibits an insulin related disorder.

7. The method of claim 6, wherein the insulin related disorder is insulin resistance; diabetes, atherosclerosis, or hypertension.

8. The method of claim 1, wherein modulating a PKC β comprises administering to the cell, tissue, or subject a PKC β agonist.

9. The method of claim 8, wherein the PKC β agonist is selected from the group of: PKCβ polypeptide or functional fragment or analog thereof; a nucleic acid sequence encoding a PKCβ polypeptide or a functional fragment or analog thereof; and an agent which increases PKCβ expression.

10. A method of increasing eNOS in a cell, tissue, or subject, comprising inhibiting a PKCβ.

11. The method of claim 10, wherein inhibiting a PKCβ comprises administering to the cell, tissue, or subject a PKCβ inhibitor.

12. The method of claim 10, wherein the inhibitor of PKCβ is selected from the group of: an inhibitory PKCβ antibody, a PKCβ antisense nucleic acid, an inhibitory PKCβ binding peptide, and an inhibitory PKCβ binding small molecule.

13. The method of claim 11, wherein the PKCβ inhibitor is LY333531.

14. The method of claim 10, wherein eNOS mRNA levels are increased.

15. The method of claim 10, wherein the subject has an insulin related disorder.

16. The method of claim 15, wherein the insulin related disorder is hypertension.

17. The method of claim 15, wherein the insulin related disorder is diabetes.

18. The method of claim 15, wherein the insulin related disorder is atherosclerosis.

19. The method of claim 15, wherein the insulin related disorder is insulin resistance.

20. A method of increasing eNOS in a cell, tissue, or subject, comprising increasing a PI3 kinase activity.

21. The method of claim 20, wherein eNOS mRNA levels are increased.

22. The method of claim 20, wherein the subject has an insulin related disorder.

23. The method of claim 22, wherein the insulin related disorder is hypertension, diabetes, atherosclerosis, ischemia, or insulin resistance.

24. A method of treating hypertension in a subject, comprising:
identifying a subject in need of treatment for hypertension; and
administering LY333531, wherein LY333531 increases eNOS expression in a tissue of the subject.

25. A method of determining if a subject is at risk for hypertension, comprising:
evaluating a PKCβ activity in a cell or tissue of the subject,
comparing the PKCβ activity in the cell or tissue of the subject to a control.

26. The method of claim 25, wherein the control is a non-hypertensive subject, or a cell or tissue therefrom.
